SEQUENCE LISTING (46 sequences)

SEQ ID NO: 1 - protein, 450 aa, synthetic construct
  REGION 1..450: note = Synthetic polypeptide
QVQLQQSGAELVRPGTSVKVSCKASGYAFINYLIEWVKQRPGQGLEWIGIISPGSGSTNYNEEFRGRATLTADRSSSTAYMQLSSLTSDDSAVYFCARGGLTHYSGSTYEEGFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 469 aa, synthetic construct
  REGION 1..469: note = Synthetic polypeptide
MEWSGVFIFLLSVTAGIHSQVQLQQSGAELVRPGTSVKVSCKASGYAFINYLIEWVKQRPGQGLEWIGIISPGSGSTNYNEEFRGRATLTADRSSSTAYMQLSSLTSDDSAVYFCARGGLTHYSGSTYEEGFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - protein, 5 aa, synthetic construct
  REGION 1..5: note = Synthetic polypeptide
NYLIE

SEQ ID NO: 4 - protein, 17 aa, synthetic construct
  REGION 1..17: note = Synthetic polypeptide
IISPGSGSTNYNEEFRG

SEQ ID NO: 5 - protein, 17 aa, synthetic construct
  REGION 1..17: note = Synthetic polypeptide
GGLTHYSGSTYEEGFDF

SEQ ID NO: 6 - protein, 30 aa, synthetic construct
  REGION 1..30: note = Synthetic polypeptide
QVQLQQSGAELVRPGTSVKVSCKASGYAFI

SEQ ID NO: 7 - protein, 14 aa, synthetic construct
  REGION 1..14: note = Synthetic polypeptide
WVKQRPGQGLEWIG

SEQ ID NO: 8 - protein, 32 aa, synthetic construct
  REGION 1..32: note = Synthetic polypeptide
RATLTADRSSSTAYMQLSSLTSDDSAVYFCAR

SEQ ID NO: 9 - protein, 11 aa, synthetic construct
  REGION 1..11: note = Synthetic polypeptide
WGQGTTLTVSS

SEQ ID NO: 10 - protein, 126 aa, synthetic construct
  REGION 1..126: note = Synthetic polypeptide
QVQLQQSGAELVRPGTSVKVSCKASGYAFINYLIEWVKQRPGQGLEWIGIISPGSGSTNYNEEFRGRATLTADRSSSTAYMQLSSLTSDDSAVYFCARGGLTHYSGSTYEEGFDFWGQGTTLTVSS

SEQ ID NO: 11 - protein, 324 aa, synthetic construct
  REGION 1..324: note = Synthetic polypeptide
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 215 aa, synthetic construct
  REGION 1..215: note = Synthetic polypeptide
QAVVTQESALTTSPGETVTLTCRSSTGAVTTTNYANWVQEKPDHLFTGLIGGTNNRAPGVPARFSGSLIGDKAALTITGAQTDDEAIYFCALWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 13 - protein, 234 aa, synthetic construct
  REGION 1..234: note = Synthetic polypeptide
MAWISLILSLLALSSGAISQAVVTQESALTTSPGETVTLTCRSSTGAVTTTNYANWVQEKPDHLFTGLIGGTNNRAPGVPARFSGSLIGDKAALTITGAQTDDEAIYFCALWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 14 - protein, 14 aa, synthetic construct
  REGION 1..14: note = Synthetic polypeptide
RSSTGAVTTTNYAN

SEQ ID NO: 15 - protein, 7 aa, synthetic construct
  REGION 1..7: note = Synthetic polypeptide
GTNNRAP

SEQ ID NO: 16 - protein, 9 aa, synthetic construct
  REGION 1..9: note = Synthetic polypeptide
ALWYSNRWV

SEQ ID NO: 17 - protein, 22 aa, synthetic construct
  REGION 1..22: note = Synthetic polypeptide
QAVVTQESALTTSPGETVTLTC

SEQ ID NO: 18 - protein, 15 aa, synthetic construct
  REGION 1..15: note = Synthetic polypeptide
WVQEKPDHLFTGLIG

SEQ ID NO: 19 - protein, 32 aa, synthetic construct
  REGION 1..32: note = Synthetic polypeptide
GVPARFSGSLIGDKAALTITGAQTDDEAIYFC

SEQ ID NO: 20 - protein, 10 aa, synthetic construct
  REGION 1..10: note = Synthetic polypeptide
FGGGTKLTVL

SEQ ID NO: 21 - protein, 109 aa, synthetic construct
  REGION 1..109: note = Synthetic polypeptide
QAVVTQESALTTSPGETVTLTCRSSTGAVTTTNYANWVQEKPDHLFTGLIGGTNNRAPGVPARFSGSLIGDKAALTITGAQTDDEAIYFCALWYSNRWVFGGGTKLTVL

SEQ ID NO: 22 - protein, 106 aa, synthetic construct
  REGION 1..106: note = Synthetic polypeptide
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 23 - DNA, 1350 bp, synthetic construct
  misc_difference 1..1350: note = Synthetic polynucleotide
  misc_difference 1..1350: note = This sequence may include a stop sequence, such as TGA
  source 1..1350: mol_type = other DNA
caggtccaactgcagcagtctggagctgaactggtaaggccggggacttcagtgaaggtgtcctgcaaggcttctggatacgccttcattaattacttgatagaatgggtgaaacagaggcctggacagggccttgagtggattggaataataagtcctggaagtggtagtactaactacaatgaagagttcaggggcagggcaacactgactgcagacagatcctccagcactgcctacatgcagctcagcagcctgacatctgatgactctgcggtctatttctgtgcaagagggggcttaactcattactccggcagtacgtacgaagagggctttgacttctggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 24 - DNA, 1407 bp, synthetic construct
  misc_difference 1..1407: note = Synthetic polynucleotide
  misc_difference 1..1407: note = This sequence may include a stop sequence, such as TGA
  source 1..1407: mol_type = other DNA
atggaatggagcggagtctttatctttctcctgtcagtaactgcaggtattcactcccaggtccaactgcagcagtctggagctgaactggtaaggccggggacttcagtgaaggtgtcctgcaaggcttctggatacgccttcattaattacttgatagaatgggtgaaacagaggcctggacagggccttgagtggattggaataataagtcctggaagtggtagtactaactacaatgaagagttcaggggcagggcaacactgactgcagacagatcctccagcactgcctacatgcagctcagcagcctgacatctgatgactctgcggtctatttctgtgcaagagggggcttaactcattactccggcagtacgtacgaagagggctttgacttctggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 25 - DNA, 15 bp, synthetic construct
  misc_feature 1..15: note = Synthetic polynucleotide
  source 1..15: mol_type = other DNA
aattacttgatagaa

SEQ ID NO: 26 - DNA, 51 bp, synthetic construct
  misc_feature 1..51: note = Synthetic polynucleotide
  source 1..51: mol_type = other DNA
ataataagtcctggaagtggtagtactaactacaatgaagagttcaggggc

SEQ ID NO: 27 - DNA, 51 bp, synthetic construct
  misc_feature 1..51: note = Synthetic polynucleotide
  source 1..51: mol_type = other DNA
gggggcttaactcattactccggcagtacgtacgaagagggctttgacttc

SEQ ID NO: 28 - DNA, 90 bp, synthetic construct
  misc_feature 1..90: note = Synthetic polynucleotide
  source 1..90: mol_type = other DNA
caggtccaactgcagcagtctggagctgaactggtaaggccggggacttcagtgaaggtgtcctgcaaggcttctggatacgccttcatt

SEQ ID NO: 29 - DNA, 42 bp, synthetic construct
  misc_feature 1..42: note = Synthetic polynucleotide
  source 1..42: mol_type = other DNA
tgggtgaaacagaggcctggacagggccttgagtggattgga

SEQ ID NO: 30 - DNA, 96 bp, synthetic construct
  misc_feature 1..96: note = Synthetic polynucleotide
  source 1..96: mol_type = other DNA
agggcaacactgactgcagacagatcctccagcactgcctacatgcagctcagcagcctgacatctgatgactctgcggtctatttctgtgcaaga

SEQ ID NO: 31 - DNA, 33 bp, synthetic construct
  misc_feature 1..33: note = Synthetic polynucleotide
  source 1..33: mol_type = other DNA
tggggccaaggcaccactctcacagtctcctca

SEQ ID NO: 32 - DNA, 378 bp, synthetic construct
  misc_difference 1..378: note = Synthetic polynucleotide
  source 1..378: mol_type = other DNA
caggtccaactgcagcagtctggagctgaactggtaaggccggggacttcagtgaaggtgtcctgcaaggcttctggatacgccttcattaattacttgatagaatgggtgaaacagaggcctggacagggccttgagtggattggaataataagtcctggaagtggtagtactaactacaatgaagagttcaggggcagggcaacactgactgcagacagatcctccagcactgcctacatgcagctcagcagcctgacatctgatgactctgcggtctatttctgtgcaagagggggcttaactcattactccggcagtacgtacgaagagggctttgacttctggggccaaggcaccactctcacagtctcctca

SEQ ID NO: 33 - DNA, 972 bp, synthetic construct
  misc_difference 1..972: note = Synthetic polynucleotide
  misc_difference 1..972: note = This sequence may include a stop sequence, such as TGA
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 34 - DNA, 645 bp, synthetic construct
  misc_difference 1..645: note = Synthetic polynucleotide
  misc_difference 1..645: note = This sequence may include a stop sequence, such as TGA
  source 1..645: mol_type = other DNA
caggctgttgtgactcaggaatctgcactcaccacatcacctggtgaaacagtcacactcacttgtcgctcaagtactggggctgttacaactactaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgatgatgaggcaatatatttctgtgctctatggtacagcaaccgctgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcc

SEQ ID NO: 35 - DNA, 702 bp, synthetic construct
  misc_difference 1..702: note = Synthetic polynucleotide
  misc_difference 1..702: note = This sequence may include a stop sequence, such as TGA
  source 1..702: mol_type = other DNA
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactactaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgatgatgaggcaatatatttctgtgctctatggtacagcaaccgctgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcc

SEQ ID NO: 36 - DNA, 42 bp, synthetic construct
  misc_feature 1..42: note = Synthetic polynucleotide
  source 1..42: mol_type = other DNA
cgctcaagtactggggctgttacaactactaactatgccaac

SEQ ID NO: 37 - DNA, 21 bp, synthetic construct
  misc_feature 1..21: note = Synthetic polynucleotide
  source 1..21: mol_type = other DNA
ggtaccaacaaccgagctcca

SEQ ID NO: 38 - DNA, 27 bp, synthetic construct
  misc_feature 1..27: note = Synthetic polynucleotide
  source 1..27: mol_type = other DNA
gctctatggtacagcaaccgctgggtg

SEQ ID NO: 39 - DNA, 66 bp, synthetic construct
  misc_feature 1..66: note = Synthetic polynucleotide
  source 1..66: mol_type = other DNA
caggctgttgtgactcaggaatctgcactcaccacatcacctggtgaaacagtcacactcacttgt

SEQ ID NO: 40 - DNA, 45 bp, synthetic construct
  misc_feature 1..45: note = Synthetic polynucleotide
  source 1..45: mol_type = other DNA
tgggtccaagaaaaaccagatcatttattcactggtctaataggt

SEQ ID NO: 41 - DNA, 96 bp, synthetic construct
  misc_feature 1..96: note = Synthetic polynucleotide
  source 1..96: mol_type = other DNA
ggtgttcctgccagattctcaggctccctgattggagacaaggctgccctcaccatcacaggggcacagactgatgatgaggcaatatatttctgt

SEQ ID NO: 42 - DNA, 30 bp, synthetic construct
  misc_feature 1..30: note = Synthetic polynucleotide
  source 1..30: mol_type = other DNA
ttcggtggaggaaccaaactgactgtccta

SEQ ID NO: 43 - DNA, 327 bp, synthetic construct
  misc_feature 1..327: note = Synthetic polynucleotide
  source 1..327: mol_type = other DNA
caggctgttgtgactcaggaatctgcactcaccacatcacctggtgaaacagtcacactcacttgtcgctcaagtactggggctgttacaactactaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgatgatgaggcaatatatttctgtgctctatggtacagcaaccgctgggtgttcggtggaggaaccaaactgactgtccta

SEQ ID NO: 44 - DNA, 318 bp, synthetic construct
  misc_difference 1..318: note = Synthetic polynucleotide
  misc_difference 1..318: note = This sequence may include a stop sequence, such as TGA
  source 1..318: mol_type = other DNA
ggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcc

SEQ ID NO: 45 - protein, 17 aa, synthetic construct
  REGION 1..17: note = Synthetic polypeptide
CQLSNRFPRYNNPEKLL

SEQ ID NO: 46 - protein, 19 aa, synthetic construct
  REGION 1..19: note = Synthetic polypeptide
ISPKTPRPGELGGRVSGSL

(19) United States
(12) Patent Application Publication
(10) Pub. No.: US 2024/0384005 A1
(43) Pub. Date: Nov. 21, 2024

(54) ANTI-NGLY-1 ANTIBODIES AND METHODS OF USE

(71) Applicant: The Jackson Laboratory, Bar Harbor, ME (US)

(72) Inventors: Alan Sawyer, Bar Harbor, ME (US); Aamir Zuberi, Bar Harbor, ME (US); Cathleen Marie Lutz, Bar Harbor, ME (US)

(73) Assignee: The Jackson Laboratory, Bar Harbor, ME (US)

(21) Appl. No.: 18/666,184

(22) Filed: May 16, 2024

Related U.S. Application Data
(62) Division of application No. 17/057,236, filed on Nov. 20, 2020, now Pat. No. 12,054,558.
(60) Provisional application No. 62/697,058, filed on Jul. 12, 2018.
(60) Provisional application No. 62/675,715, filed on May 23, 2018.

(51) Int. Cl. C07K 16/40; A61K 39/00; G01N 33/573

(52) U.S. Cl. CPC C07K 16/40; G01N 33/573; A61K 2039/505; C07K 2317/34; C07K 2317/565; C07K 2317/567; C07K 2317/92; G01N 2800/52

(57) ABSTRACT

Provided herein, in some embodiments, are anti-N-glycanase 1 antibodies and methods of use.

### RELATED APPLICATIONS

This application is a divisional of U.S. application Ser. No. 17/057,236, filed Nov. 20, 2020, which is a national stage filing under 35 U.S.C. § 371 of International Patent Application Serial No. PCT/US2019/033499, filed May 22, 2019, which was published under PCT Article 21(2) in English and claims the benefit under 35 U.S.C. § 119(e) of U.S. provisional application No. 62/675,715, filed May 23, 2018, and U.S. provisional application No. 62/697,058, filed Jul. 12, 2018, each of which is incorporated by reference herein in its entirety.

### REFERENCE TO AN ELECTRONIC SEQUENCE LISTING

The content of the electronic sequence listing (J022770013US03-SUBSEQ-HJD.txt; Size: 69,912 bytes; and Date of Creation: Jun. 5, 2024) is herein incorporated by reference in its entirety.

### BACKGROUND

The Ngly1R539X allele has a one (1) base pair replacement in the mouse N-glycanase 1 (NGly-1) gene that encodes a nonsense mutation, resulting in a premature stop codon. This mutation corresponds to the R524X mutation identified in the patient population as a disease causing variant in Congenital Disorders of Glycosylation Type IV, also referred to as Alacrimia-Choreoathetosis-Liver Dysfunction Syndrome and NGLY1 deficiency.

### SUMMARY

Provide herein, in some embodiments, is an antibody that binds specifically to mouse NGly-1 and human NGLY-1. In some embodiments, the antibody comprises a heavy chain comprising, (a) a CDR1 comprising the sequence of SEQ ID NO: 3, (b) a CDR2 comprising the sequence of SEQ ID NO: 4, and (c) a CDR3 comprising the sequence of SEQ ID NO: 5. In some embodiments, the anti-NGly-1 antibody comprises a light chain comprising, (a) a CDR 1 comprising the sequence of SEQ ID NO: 14, (b) a CDR2 comprising the sequence of SEQ ID NO: 15, and (c) a CDR3 comprising the sequence of SEQ ID NO: 16.

Also provided herein are compositions and kits comprising an antibody that binds specifically to NGly-1 and NGLY-1.

Further provided herein are methods that include contacting a cell with an antibody that binds specifically to NGly-1 and NGLY-1. Such methods may be used, for example, to diagnose a subject with NGLY1 deficiency. Diagnostic kits comprising the antibodies described herein are also provided.

Also provided herein are methods for using an antibody that binds specifically to NGly-1 and NGLY-1 for the treatment of a cancer.

The antibodies provided herein can bind specifically to either human NGLY-1 protein or mouse NGly-1 protein. For simplicity, it should be understood that reference to an “antibody that binds to specifically NGly-1” or an “anti-Ngly-1 antibody” encompasses antibodies that can bind specifically to mouse NGly-1 protein and can bind specifically to human NGLY-1 protein.

### BRIEF DESCRIPTION OF THE DRAWINGS

FIG. 1 shows an antigen index plot (top), a hydrophilicity plot (middle), and a surface probability plot (bottom) of NGly-1 amino acid sequences 283 to 299 (SEQ ID NO: 45).

FIG. 2 shows an antigen index plot (top), a hydrophilicity plot (middle), and a surface probability plot (bottom) of NGly-1 amino acid sequences 442 to 460 (SEQ ID NO: 46).

FIG. 3 shows the structure of full length NGLyc-1 with two candidate peptides selected.

FIG. 4 shows data demonstrating anti-NGly-1 specificity of the monoclonal antibody of the present disclosure. Tissues were collected from embryonic E16.5 day brain and heart using C57BL/6J (WT) and JR 27060 Ngly-1 null homozygous tissues (HOM).

FIG. 5 shows data demonstrating that the mouse anti-NGly-1 monoclonal antibody of the present disclosure cross-reacts with human NGLY-1. Lane 1: F47E6 Mouse C57BL/6J (WT); Lane 2: F47E1 27060 Mouse Ngly-1 null mutant; Lane 3: Human iPSC cell: DGW-A1 (NGLY 1 double mutant)-derived from an affected patient; Lane 4: Human iPSC cell: 39B9-1 (one allele of the double mutant of NGLY-1 corrected); Lane 5: Human H9 Wild-type ES cells.

FIG. 6 shows data demonstrating that one of the publicly-available anti-NGly-1 antibodies detects a cross reactive protein that is very similar in size to the native mouse NGly-1 protein, indicating that the antibody is not specific for NGly-1. The tissues used were isolated from B57BL/6J (JR 00664) and homozygous Ngly 1 null (JR 27060) embryos, aged E16.5 days.

FIG. 7 shows data demonstrating that a commercially-available anti-NGly-1 antibody (SIGMA) is not specific for NGly-1. Predominant bands of the expected size are seen in Lanes 2, 6, 9, 10, 13 of Panel A and Lane 1 of Panel B. Panel A-Lane 1: JR00664 Heart Total Extract; Lane 2: JR 00664 Heart Mitochondrial Fraction; Lane 3: JR 00664 Heart Cytosolic Fraction; Lane 4: JR00664 Kidney Total extract; Lane 5: JR 00664 Kidney Mitochondrial Fraction; Lane 6: JR 00664 Kidney Cytosolic Fraction; Lane 7: JR00664 Liver Total Extract; Lane 8: JR27962 Liver Mitochondrial Fraction; Lane 9: JR 00664 Liver Mitochondrial Fraction; Lane 10: JR00664 Liver Cytosolic Fraction; Lane 11: JR 00664 Skeletal Muscle Mitochondrial Fraction; Lane 12: JR 00664 Skeletal Muscle Mitochondrial Fraction; Lane 13: JR 00664 Skeletal Muscle Cytosolic Fraction. Panel B-Lane 1: C2C12 Mouse myocyte cell line; Lane 2: 2H11 Mouse endothelial cell line; Lane 3: mK4 Human embryonic kidney cell line; Lane 4: NIH 3T3 Mouse fibroblast cell line; Lane 5: JR 00664 Brain total extract; Lane 6: No lysate control. Tissue was obtained from a JR00664 C57BL/6J mouse strain and JR27962 C57BL/6J-Ngly1em9Lutzy/J (an Ngly1 deficient) mouse strain.

FIGS. 8A and 8B show data demonstrating detection of a 62 kDa polypeptide in mouse C57BL/6J tissues using another commercially-available anti-NGly-1 antibody (ABCAM). This is a smaller protein than observed using the anti-NGly-1 monoclonal antibody of the present disclosure. Further, a strong cross-reactivity was detected against a small 2-3 kDa polypeptide using the commercially-available anti-NGly-1 antibody, and no expression of the 62 kDa protein was detected in brain tissue. This suggests that the protein(s) detected with this commercially-available antibody may not be mouse NGly-1. FIG. 8B: Lane 1: JR00664 Brain Total extract; v2: JR 00664 Brain Mitochondrial fraction; Lane 3: JR 00664 Brain Cytosolic Fraction; Lane 4: JR00664 Liver Total extract; Lane 5: JR 27962 (Het) Liver Total extract; Lane 6: JR 00664 Liver Mitochondrial Fraction; Lane 7: JR 00664 Liver cytosolic fraction; Lane 8: NIH3T3 cell line (mouse).

FIG. 9 shows data demonstrating that yet another commercially-available anti-NGlyc-1 antibody (ENCOR) produces a high background, and the predominant protein detected is significantly smaller than expected. Screening the antibody against lysates from known Ngly-1 protein null strains revealed multiple bands remaining. Thus, this commercially-available antibody is non-specific. The antibody was made against the N-terminal 300 amino acids of Human NGLY-1. Lane 1: JR00664 C57BL/6J E16.5 embryo Brain; Lane 2: JR27060 Ngly 1 null mouse E16.5 embryo Brain; Lane 3: JR28975 Ngly 1 null mouse E16.5 embryo Brain.

### DETAILED DESCRIPTION

NGLY1 deficiency is a rare inherited disorder that affects children lacking NGLY1 normal expression or function. These children demonstrate global development delay, loss of muscle tone, peripheral neuropathy, seizures, poor reflexes, speech impairment, lack of tear production, corneal scarring, chronic constipation and/or liver dysfunction. The protein product of NGLY1 encodes an enzyme called N-glycanase 1, also known as PNGase, which catalyzes the removal of N-linked oligosaccharides from the asparagine side chains of glycoproteins. Cytoplasmic PNGase activity was first reported in Saccharomyces cerevisiae (Suzuki et al 2000), but subsequently found to be evolutionarily conserved in many species including Drosophila, C. elegans, mouse and human.

Some aspects of the present disclosure provide an antibody that binds specifically to NGly-1 and comprises a heavy chain and a light chain, wherein the heavy chain comprises (a) a CDR1 comprising the sequence of SEQ ID NO: 3, (b) a CDR2 comprising the sequence of SEQ ID NO: 4, and (c) a CDR3 comprising the sequence of SEQ ID NO: 5, and/or the light chain comprises (a) a CDR1 comprising the sequence of SEQ ID NO: 14, (b) a CDR2 comprising the sequence of SEQ ID NO: 15, and (c) a CDR3 comprising the sequence of SEQ ID NO: 16.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv), single chain variable fragment (scFv), mutants thereof, fusion proteins comprising an antibody portion, humanized antibodies, chimeric antibodies, diabodies, linear antibodies, single chain antibodies, single domain antibodies (e.g., camel or llama VHH antibodies), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

In some embodiments, an antibody of the present disclosure is a monoclonal antibody. A “monoclonal antibody” refers to a homogenous antibody population.

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In other embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen, such as (mouse or human) NGly-1. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to an NGly-1 epitope is an antibody that binds this NGly-1 epitope with greater affinity, avidity, more readily, and/or with greater duration than it binds to other NGly-1 epitopes or non-NGly-1 epitopes. In some embodiments, the NGly-1 epitope comprises SEQ ID NO: 45 or 46. In some embodiments, the disclosure provides a peptide comprising or consisting of the amino acid sequence of SEQ ID NO: 45. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, the equilibrium dissociation constant (K_{D}) between the antibody and NGly-1 is 100 pM to 1000 pM. For example, the K_{D} between the antibody and NGly-1 may be 100 pM to 900 pM, 100 pM to 800 pM, 100 pM to 700 pM, 100 PM to 600 pM, 100 pM to 500 pM, 100 pM to 400 pM, 100 pM to 300 pM, 150 pM to 1000 pM, 150 pM to 900 pM, 150 pM to 800 pM, 150 pM to 700 pM, 150 pM to 600 pM, 150 pM to 500 pM, 150 pM to 400 pM, 150 pM to 300 pM, 200 pM to 1000 pM, 200 pM to 900 pM, 200 pM to 800 pM, 200 pM to 700 pM, 200 pM to 600 pM, 200 pM to 500 pM, 200 pM to 400 pM, 200 pM to 300 pM, 250 pM to 1000 pM, 250 pM to 900 pM, 250 pM to 800 pM, 250 pM to 700 pM, 250 pM to 600 pM, 250 pM to 500 pM, 250 pM to 400 pM, or 250 pM to 300 pM. In some embodiments, the equilibrium dissociation constant (K_{D}) between the antibody and NGly-1 is 100 pM, 150 pM, 200 pM, 210 pM, 220 pM, 230 PM, 240 PM, 250 pM, 260 pM, 270 pM, 280 pM, 290 pM, or 300 pM. In some embodiments, the equilibrium dissociation constant (K_{D}) between the antibody and NGly-1 is less than 500 pM, less than 400 pM, or less than 300 pM. In some embodiments, the K_{D} between the antibody and NGly-1 is 1 nM to 100 nM.

### Heavy Chain

A heavy chain is the large polypeptide subunit of an antibody. Heavy chains differ in size and composition, but are typically between 450 and 550 amino acids in length and are composed of a constant domain (HC Constant), comprising three or four immunoglobulin domains, and a variable domain (HC Variable), comprising a single immunoglobulin domain. The variable domain of the heavy chain is important for binding antigen. An immunoglobulin domain is a structure formed by the three-dimensional arrangement of beta-strands into parallel beta-sheets. There are five types of heavy chains in mammals which define the class of antibody, wherein IgA antibodies contain alpha (α) heavy chains, IgD antibodies contain delta (δ) heavy chains, IgE antibodies contain epsilon (ε) heavy chains, IgM antibodies contain mu (μ) heavy chains, and IgG antibodies contain gamma (γ) heavy chains.

In some embodiments, the heavy chain of an antibody of the present disclosure comprises the sequence of SEQ ID NO: 1. In some embodiments, the heavy chain of an antibody of the present disclosure comprises a sequences that has at least 95%, 96%, 97%, 98%, or 99% identity to the sequence of SEQ ID NO: 1.

### Light Chain

A light chain is the small polypeptide of an antibody. Light chains differ in size, but are typically between 210 and 217 amino acids in length and are composed of a constant domain (LC Constant), comprising a single immunoglobulin domain, and a variable domain (LC Variable), comprising a single immunoglobulin domain. There are two types of light chains in mammals, which are defined by the sequence of the constant region and classified as either kappa (κ) or lambda (λ). The variable domain of the light chain is important for binding antigen. Only one type of light chain is typically present in an antibody, so the two light chains within a single antibody are identical.

In some embodiments, the light chain of an antibody of the present disclosure comprises the sequence of SEQ ID NO: 13. In some embodiments, the light chain of an antibody of the present disclosure comprises a sequences that has at least 95%, 96%, 97%, 98%, or 99% identity to the sequence of SEQ ID NO: 13.

### Variable Region

Each antibody has a unique variable region composed of the variable domains of both heavy and light chains which contains the antigen binding site. The variable region is further subdivided into complementarity determining regions (CDRs) and framework (FR) regions. There are two variable domains on each antibody which typically, but not always, bind the same antigen.

In some embodiments, the heavy chain comprises a variable region that is at least 90% or at least 95% (e.g., at least 96%, 97%, 98%, or 99%) identical to the sequence of SEQ ID NO: 10. In some embodiments, the heavy chain comprises a variable region that comprises the sequence of SEQ ID NO: 10.

In some embodiments, the light chain comprises a variable region that is at least 90% or at least 95% (e.g., at least 96%, 97%, 98%, or 99%) identical to the sequence of SEQ ID NO: 21. In some embodiments, the light chain comprises a variable region that comprises the sequence of SEQ ID NO: 21.

### CDR

A complementarity-determining region (CDR) (also known as a hypervariable region, or HV) within the variable region has a high ratio of different amino acids in a given position, relative to the most common amino acid in that position. Three CDRs (CDR1, CDR2, CDR3) exist within heavy and light chains, which form flexible loops that directly contact a portion of the antigen's surface.

In some embodiments, an antibody that binds specifically to NGly-1 comprises a heavy chain comprising (a) a CDR1 comprising the sequence of SEQ ID NO: 3, (b) a CDR2 comprising the sequence of SEQ ID NO: 4, and (c) a CDR3 comprising the sequence of SEQ ID NO: 5.

In some embodiments, an antibody that binds specifically to NGly-1 comprises a light chain comprising (a) a CDR1 comprising the sequence of SEQ ID NO: 14, (b) a CDR2 comprising the sequence of SEQ ID NO: 15, and (c) a CDR3 comprising the sequence of SEQ ID NO: 16.

### Framework Region

The framework region (FR) within a variable region is composed of conserved amino acid sequences which separate CDR sequences. The FR regions form a beta-sheet structure which serves as a scaffold to hold the CDRs in position to contact the antigen surface. Four FR regions exist within each heavy and light chain.

In some embodiments, the heavy chain comprises a framework region that is at least 90% or at least 95% (e.g., at least 96%, 97%, 98%, or 99%) identical to the sequence of SEQ ID NO: 6; a framework region that is at least 90% or at least 95% (e.g., at least 96%, 97%, 98%, or 99%) identical to the sequence of SEQ ID NO: 7; a framework region that is at least 90% or at least 95% identical to the sequence of SEQ ID NO: 8; and/or a framework region that is at least 90% or at least 95% (e.g., at least 96%, 97%, 98%, or 99%) identical to the sequence of SEQ ID NO:9.

In some embodiments, the light chain comprises a framework region that is at least 90% (e.g., at least 91%, 92%, 93%, 94%, 95%, 96%, 97%, 98%, or 99%) identical to the sequence of SEQ ID NO: 17; a framework region that is at least 90% (e.g., at least 91%, 92%, 93%, 94%, 95%, 96%, 97%, 98%, or 99%) identical to the sequence of SEQ ID NO: 18; a framework region that is at least 90% (e.g., at least 91%, 92%, 93%, 94%, 95%, 96%, 97%, 98%, or 99%) identical to the sequence of SEQ ID NO: 19; and/or a framework region that is at least 90% (e.g., at least 91%, 92%, 93%, 94%, 95%, 96%, 97%, 98%, or 99%) identical to the sequence of SEQ ID NO: 20.

### Constant Region

The constant region of an antibody is recognized by receptors on immune cells and proteins to initiate and regulate host defense mechanisms. The constant region of heavy chain polypeptides is identical in all antibodies of the immunoglobulin class, but differs between immunoglobulin classes. Heavy chains in Igγ, Igα, and Igδ contain a constant region composed of three immunoglobulin domains and a hinge region for increased flexibility. Heavy chains in Igμ and Igε contain a constant region composed of four immunoglobulin domains. The constant region of light chain polypeptides is composed of a single immunoglobulin domain.

In some embodiments, a heavy chain comprises a constant region that is at least 90% or at least 95% (e.g., at least 96%, 97%, 98%, or 99%) identical to the sequence of SEQ ID NO: 11. In some embodiments, a heavy chain comprises a constant region that comprises the sequence of SEQ ID NO: 11.

In some embodiments, the light chain comprises a constant region that is at least 90% or at least 95% (e.g., at least 96%, 97%, 98%, or 99%) identical to the sequence of SEQ ID NO: 22. In some embodiments, a light chain comprises a constant region that comprises the sequence of SEQ ID NO: 22.

### Polynucleotides and Antibody Synthesis

The present disclosure also provides polynucleotides encoding the antibody described herein. “Polynucleotide,” as used herein, includes both a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. An “isolated” polynucleotide is, for example, DNA or RNA that has been removed from its native environment. For example, a recombinant polynucleotide encoding an antibody contained in a vector is considered isolated for the purposes of the present disclosure. Further examples of isolated polynucleotides include recombinant polynucleotides maintained in heterologous host cells or purified (partially or substantially) polynucleotides in solution. Isolated RNA molecules include in vivo or in vitro RNA transcripts of polynucleotides of the present disclosure. Isolated polynucleotides or nucleic acids may further include such molecules produced synthetically. In addition, a polynucleotide may include a regulatory element such as a promoter, ribosome binding site, or a transcription terminator. Exemplary polynucleotide sequences are provided in Table 2 (SEQ ID NOs: 23-44).

Following synthesis of the polynucleotide encoding the antibody, the polynucleotide may be inserted in an expression vector for introduction into host cells that may be used to produce the desired antibody. Recombinant expression of an antibody, or fragment thereof, e.g., a heavy or light chain of an antibody is described herein. Once a polynucleotide encoding an antibody molecule or a heavy or light chain of an antibody, or portion thereof (preferably containing the heavy or light chain variable domain), has been obtained, the vector for the production of the antibody molecule may be produced by recombinant DNA technology using techniques available in the art. Thus, methods for preparing a protein by expressing a polynucleotide containing an antibody encoding nucleotide sequence are described herein. Methods that are available to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The disclosure therefore provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule, and the variable domain of the antibody may be cloned into such a vector for expression of the entire heavy or light chain.

The term “vector” or “expression vector” is used herein to refer to a vehicle for introducing into and expressing a desired gene in a host cell. Vectors may be selected from the group consisting of plasmids, phages, viruses and retroviruses. In some embodiments, vectors comprise a selection marker, appropriate restriction sites to facilitate cloning of the desired gene, and the ability to enter and/or replicate in eukaryotic or prokaryotic cells. Numerous expression vector systems may be employed in accordance with the disclosure.

Once the vector or DNA sequence encoding a monomeric subunit of the antibody has been prepared, the expression vector may be introduced into an appropriate host cell. Introduction of the plasmid into the host cell can be accomplished by various techniques available to those of skill in the art. These include, but are not limited to, transfection including lipotransfection using, e.g., Fugene® or lipofectamine, protoplast fusion, calcium phosphate precipitation, cell fusion with enveloped DNA, microinjection, and infection with intact virus. In some embodiments, plasmid introduction into the host is via standard calcium phosphate co-precipitation method. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), and immunohistochemistry.

The expression vector may be transferred to a host cell by any available cell-transfection technique. The transfected cells may then be cultured by available techniques to produce an antibody described herein. Thus, the disclosure provides host cells containing a polynucleotide encoding an antibody of the present disclosure, or a heavy or light chain thereof, in some embodiments, operably linked to a heterologous promoter.

Once an antibody molecule described herein has been recombinantly expressed, the whole antibody, a dimer, an individual light and/or heavy chain, or other immunoglobulin form, may be purified according to available purification procedures, including for example, by chromatography (e.g., ion exchange, affinity, particularly by affinity for the specific antigen after Protein A, and sizing column chromatography), centrifugation, differential solubility, e.g. ammonium sulfate precipitation, or by any other standard technique for the purification of proteins; see, e.g., Scopes, “Protein Purification”, Springer Verlag, N.Y. (1982).

### Detection of N-glycanase 1 (NGLY-1) and Diagnosis of NGLY-1 Deficiency

Another aspect of the present disclosure provides methods of detecting N-glycanase 1 (NGLY-1) and diagnosing NGLY-1 deficiency using an antibody described herein. These methods may also be used to monitor the efficacy of a NGLY-1 deficiency therapy. As the anti-NGLY-1 antibodies described herein have strong binding affinity for NGLY-1 and are useful for detecting the presence of NGLY-1 in a biological sample, a lack of anti-NGLY-1 antibody binding, or binding of the anti-NGLY-1 antibody below a particular threshold relative to a control, is indicative of NGLY-1 deficiency. The term “detecting”, as used herein, refers to quantitative and/or qualitative detection.

In some embodiments, an anti-NGLY-1 antibody for use in a method of diagnosis or detection is provided. For example, a method of detecting the presence (or absence) of NGLY-1 in a biological sample is provided. In some embodiments, a method comprises contacting the biological sample with an anti-NGLY-1 antibody as described herein under conditions permissive for binding of the anti-NGLY-1 antibody to NGLY-1, and detecting whether a complex is formed between the anti-NGLY-1 antibody and NGLY-1. Such method may be performed in vitro, for example. The presence of the anti-NGLY-1 antibody-NGLY-1 complex may be indicative of a healthy subject (at a subject who does not have a NGLY-1 deficiency), whereas a lack of complex formation may indicate that the subject has a NGLY-1 deficiency.

Likewise, the level of NGLY-1 may be monitored over time, for example, to determine the efficacy of an NGLY-1 deficiency therapy. Monitoring may take place at evenly-spaced intervals, for example daily, every 2 days, every 3 days, every 4 days, every 5 days, every 6 days, weekly, biweekly, every third week, monthly, bimonthly, every 3^{rd} month, every 4^{th} month, every 5^{th} month, every 6^{th} month, every 7^{th} month, every 8^{th} month, every 9 month, every 10^{th} month, every 11^{th} month, or annually. In some embodiments, the monitoring occurs at differently-spaced intervals. For example, if a subject's NGLY-1 levels increase over a monitoring period, less frequent monitoring may be required. Conversely, if a subject's NGLY-1 levels decrease over a monitoring period, more frequent monitoring may be required. In some embodiments, the level of NGLY-1 present in a subject's sample may be compared to that of a reference sample. A “reference sample,” as used herein, refers to a sample from an individual who does not have an NGLY-1 deficiency or a sample from an individual who does have an NGLY-1 deficiency. As an example, efficacious treatment may be indicated when a subject undergoing an NGLY-1 deficiency treatment having an NGLY-1 level greater than that of the reference sample from a subject who has an NGLY-1 deficiency may be indicative of efficacious treatment.

Detection of the anti-NGLY-1 antibody binding may be performed using any method available in the art, including immunoassays, including, for example, immunohistochemistry (IHC), immunofluorescence (IF), immunoblotting (e.g., Western blotting), flow cytometry (e.g., FACS™), and Enzyme-linked Immunosorbant Assay (ELISA). In some embodiments, immunoblotting is used for detection.

Therefore, in some embodiments, the antibody is conjugated to a detectable label (antibody-label conjugate), such as an enzyme, radioisotope, fluorophore, heavy metal (colloidal metals), or luminescent molecule. Examples of detectable labels include, but are not limited to, luciferase, green fluorescent protein (GFP), chloramphenicol acetyl transferase (CAT), ³²P, ³³P, ¹⁴C, ¹²⁵I, ¹³¹I, ³⁵S, ³H, fluorescein, fluorescein isothiocyanate (FITC), DyLight 488, phycoerythrin (PE), propidium iodide (PI), PerCP, PE-Alexa Fluor® 700, Cy5, allophycocyanin, Cy7, any of a variety of luminescent lanthanide (e.g., europium or terbium) chelates (e.g., the europium chelate of diethylene triamine pentaacetic acid (DTPA) or tetraazacyclododecane-1,4,7,10-tetraacetic acid (DOTA)), alkaline phosphatase, horseradish peroxidase, gold, copper, mercury, and cadmium. The detectable label(s) may be incorporated into the anti-NGLY-1 antibody as fusion proteins, in some embodiments. Methods for generating nucleic acids encoding an antibody-heterologous polypeptide fusion protein are well known in the art of antibody engineering and described in, e.g., Dakappagari et al. (2006) J Immunol 176:426-440.

The disclosure further provides for the use of an anti-NGLY-1 antibody in a method of diagnosing a subject suffering from a disorder (e.g., NGLY-1 deficiency), the method comprising: determining the presence or expression level of NGLY-1 in a sample obtained from the subject by contacting the sample with an anti-NGLY-1 antibody described herein and detecting the presence of the bound antibody. The biological sample refers to a subset of tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen) from a subject. A sample further can include a homogenate, lysate or extract prepared from a subject or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs.

The disclosure yet further provides for the use of an anti-NGLY-1 antibody in the manufacture of a reagent for use in a method of diagnosing a subject suffering from a disorder (e.g., NGLY-1 deficiency), the method comprising: determining the presence or expression level of NGLY-1 in a sample obtained from the subject by contacting the sample with an anti-NGLY-1 antibody of the disclosure and detecting the presence of the bound antibody.

Provided herein are kits which include the diagnostic anti-NGLY-1 antibodies described herein and instructions for use. Accordingly, in some embodiments, the kit comprises an antibody comprising the heavy and light chain variable region CDR sequences set forth in SEQ ID NOs: 3-5 and 14-16, respectively, heavy and light chain variable region sequences set forth in SEQ ID NOs: 10 and 21, respectively, wherein the antibody or antigen-binding fragment thereof specifically binds to human NGLY-1, or full length heavy and light chain sequences set forth in SEQ ID NOs: 1 and 2, respectively. In certain embodiments, the antibody comprises a detectable label, e.g., an antibody-label conjugate. Such kits may comprise at least one additional reagent. For example, in some embodiments, the kits comprise buffers, stabilizers, substrates, at least one immunodetection reagent (e.g., secondary antibodies for use in immunohistochemistry), and/or cofactors required for the assay. In some embodiments, the antibody and, optionally the reagents, are suitably aliquoted. In some embodiments, the kit comprises a means for obtaining the biological sample from a subject. Such means can comprise, for example, reagents that can be used to obtain fluid or tissue sample from the subject.

### Pharmaceutical Compositions

The disclosure, in some aspects, provides pharmaceutical compositions comprising an antibody described herein and a pharmaceutically-acceptable carrier. A “pharmaceutically acceptable carrier,” after administration to or upon a subject (e.g., a human), does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the composition further comprises an additional agent for treating an NGLY-1 deficiency, for example, enzyme replacement therapy, and/or proton pump inhibitors (ENGase inhibitors) (Bi et al., Bioorg Med Chem Lett. 2017;27:2962-6).

In some embodiments, the composition is a diagnostic composition and further comprises at least one reagent for use the immune-and/or nucleic acid-based diagnostic methods described herein. For example, the reagents includes, but are not limited to, those used in immunoassays, including, for example, immunohistochemistry (IHC), immunofluorescence (IF), immunoblotting (e.g., Western blotting), flow cytometry (e.g., FACS™), and Enzyme-linked Immunosorbant Assay (ELISA). Such reagents are specific to the type of diagnostic method used, and are known in the art.

### Methods of Treatment

In some embodiments, the present disclosure provides methods of treating a disease or disorder in a patient comprising administering to the patient in need thereof a therapeutically effective amount of an antibody that binds NGLY-1 protein. In some embodiments, the disease or disorder is cancer. The cancer may be a carcinoma, sarcoma, lymphoma, leukemia, melanoma, mesothelioma, multiple myeloma, or seminoma or the cancer may be melanoma, ovarian cancer, breast cancer, cervical cancer, endometrial cancer, colon cancer, pancreatic cancer, prostate cancer, liver cancer, renal cancer, lung cancer, glioma, or lymphoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is glioma, liver cancer, or melanoma. The cancer, in some embodiments, is a melanoma.

In some embodiments, the methods further comprise administering a second agent/cancer therapy such as surgery, a second chemotherapeutic agent, a radiotherapy, or an immunotherapy. The second chemotherapeutic agent, in some embodiments, is an alkylating agent or an alkylating-like agent such as cisplatin, dacarbazine, or temozolomide.

In some embodiments, a pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, a pharmaceutical composition is formulated as a unit dose.

A pharmaceutical composition may be administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by available pharmaceutical procedures. Dosage for any one subject depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation.

### EXAMPLES

### Example 1

The protein sequence of Mouse Ngly1 was obtained from the STRING interaction networks database (string-db.org). The sequence was loaded into the DNASTAR software package using the EditSeq application. The EditSeq file was then loaded into the Protean application to generate a plot of specific physico-chemical properties:
1) Antigenic Index using the Jameson Wolf algorithm
2) Hydrophilicity using the Kyle Doolittle algorithm
3) Surface probability plot using the Emini algorithm

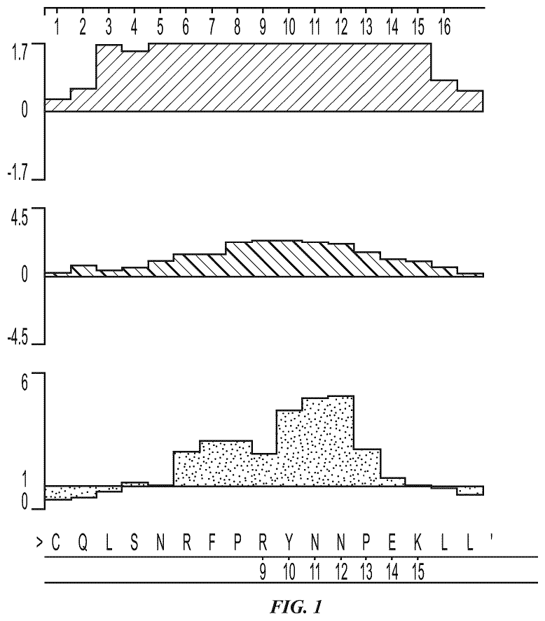

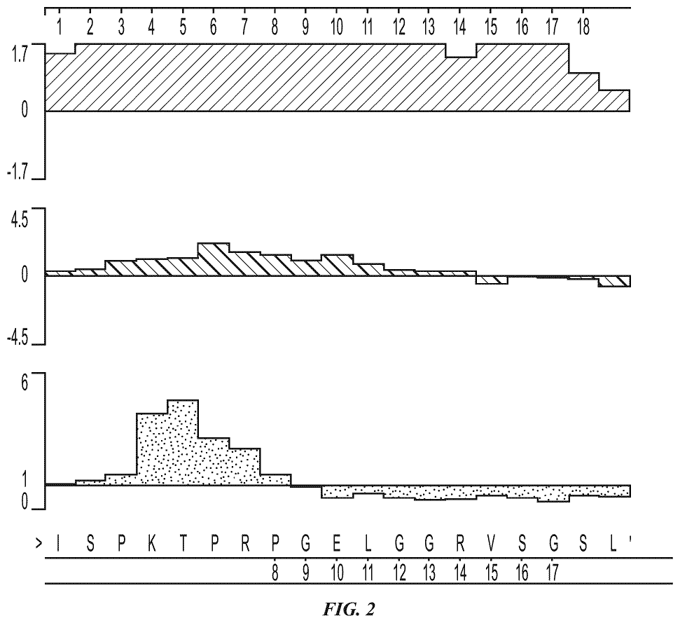

Two peptide candidates were selected for review in 3-D models based on high antigenic index, high hydrophilicity and high surface probability representing amino acid stretches 283-299 (FIGS. 1) and 442-460 (FIG. 2).

3-D models were generated using the Max Planck Institute's MPI Bionformatics Toolkit, specifically the HHpred modeling tool. Atomic coordinates from this were loaded into a local copy of MacPyMol 3-D modeling and rendering software and a 3-D model generated.

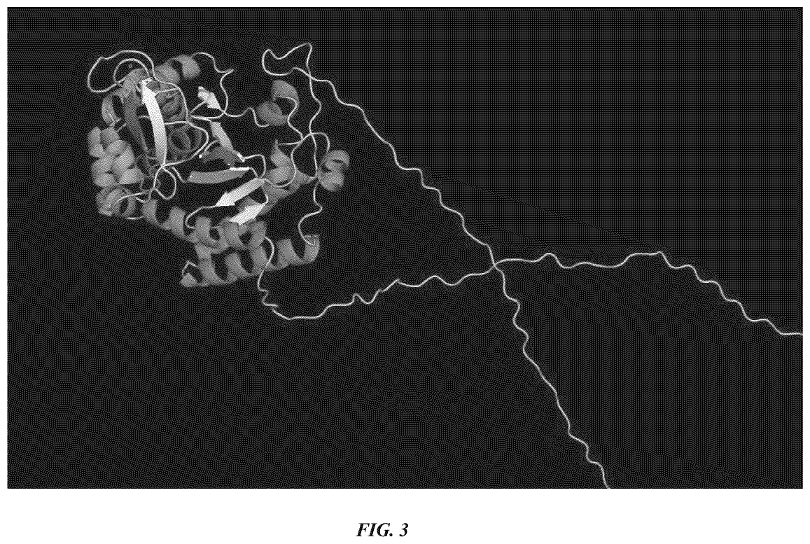

Peptide 1, a 17-mer amino acid peptide CQLSNRFPRYNNPEKLL (SEQ ID NO: 45) derived from murine Ngly1 amino acids 283-299, inclusive, was selected and highlighted in magenta; and Peptide 2 (SEQ ID NO: 46) representing amino acids 442 to 460 was selected and highlighted in cyan (FIG. 3).

Peptide 2 was rejected as a peptide candidate due to it being structured and relatively inaccessible. Peptide 1 showed a disordered region with high accessibility and synthesis of this peptide was ordered through Peptide Specialty Labs GmbH in Heidelberg, Germany. The peptide sequence is identical to the syntenic region in human NGLY1, except for a single polymorphism in the third amino acid. The amino acid is leucine in the mouse and phenylalanine in human. A portion of the peptide was conjugated to the diphtheria-toxoid carrier protein for immunization.

The conjugate was injected into female BALB/cJ mice at a dose of 10 μg per mouse with Alum adjuvant. Mice were immunized by i.p injection three times approximately two (2) weeks apart. Ten days after the third immunization, splenocytes were isolated and fused with SP2 myeloma cells to create hybridoma cell lines. Hybridomas were cultured in 96-well plates and supernatants screened on protein arrays and by ELISA. Positive clones were further validated by western blot using lysates prepared from C57BL/6J and the CRISPR/Cas9 generated null strain as positive and negative controls. Additional immunoblots were used to identify additional clones that generated antibodies capable of cross reacting with human NGLY1 protein. Isogenic human derived NGLY1 null and wild type iPS cell lines providing positive and negative controls evaluation of anti-human NGLY1 cross-reactivity were provided. A single hybridoma gave rise to clone 5G8 that generated an antibody that was specific to both murine and human NGLY1 protein and was used for subsequent studies.

TABLE 1
Anti-NGly-1 Antibody Amino Acid Sequences

| Antibody | | SEQ ID |
|---|---|---|
| Component | Amino Acid Sequence | NO: |
| Heavy Chain | QVQLQQSGAELVRPGTSVKVSCKASGYAFINYLIEWVKQRPGQGLEWIG | 1 |
| | IISPGSGSTNYNEEFRGRATLTADRSSSTAYMQLSSLTSDDSAVYFCARG | |
| | GLTHYSGSTYEEGFDFWGQGTTLTVSSAKTTPPSVYPLAPGSAAQTNSM | |
| | VTLGCLVKGYFPEPVTVTWNSGSLSSGVHTFPAVLQSDLYTLSSSVTVPS | |
| | STWPSETVTCNVAHPASSTKVDKKIVPRDCGCKPCICTVPEVSSVFIFPPK | |
| | PKDVLTITLTPKVTCVVVDISKDDPEVQFSWFVDDVEVHTAQTQPREEQF | |
| | NSTFRSVSELPIMHQDWLNGKEFKCRVNSAAFPAPIEKTISKTKGRPKAP | |
| | QVYTIPPPKEQMAKDKVSLTCMITDFFPEDITVEWQWNGQPAENYKNTQ | |
| | PIMDTDGSYFVYSKLNVQKSNWEAGNTFTCSVLHEGLHNHHTEKSLSHS | |
| | PGK | |
| Heavy Chain | MEWSGVFIFLLSVTAGIHSQVQLQQSGAELVRPGTSVKVSCKASGYAFIN | 2 |
| with leader | YLIEWVKQRPGQGLEWIGIISPGSGSTNYNEEFRGRATLTADRSSSTAYM | |
| sequence | QLSSLTSDDSAVYFCARGGLTHYSGSTYEEGFDFWGQGTTLTVSSAKTTP | |
| | PSVYPLAPGSAAQTNSMVTLGCLVKGYFPEPVTVTWNSGSLSSGVHTFP | |
| | AVLQSDLYTLSSSVTVPSSTWPSETVTCNVAHPASSTKVDKKIVPRDCGC | |
| | KPCICTVPEVSSVFIFPPKPKDVLTITLTPKVTCVVVDISKDDPEVQFSWFV | |
| | DDVEVHTAQTQPREEQFNSTFRSVSELPIMHQDWLNGKEFKCRVNSAAF | |
| | PAPIEKTISKTKGRPKAPQVYTIPPPKEQMAKDKVSLTCMITDFFPEDITVE | |
| | WQWNGQPAENYKNTQPIMDTDGSYFVYSKLNVQKSNWEAGNTFTCSV | |
| | LHEGLHNHHTEKSLSHSPGK | |
| VH CDR1 | NYLIE | 3 |
| VH CDR2 | IISPGSGSTNYNEEFRG | 4 |
| VH CDR3 | GGLTHYSGSTYEEGFDF | 5 |
| VH FR1 | QVQLQQSGAELVRPGTSVKVSCKASGYAFI | 6 |
| VH FR2 | WVKQRPGQGLEWIG | 7 |
| VH FR3 | RATLTADRSSSTAYMQLSSLTSDDSAVYFCAR | 8 |
| VH FR4 | WGQGTTLTVSS | 9 |
| HC Variable | QVQLQQSGAELVRPGTSVKVSCKASGYAFINYLIEWVKQRPGQGLEWIG | 10 |
| | IISPGSGSTNYNEEFRGRATLTADRSSSTAYMQLSSLTSDDSAVYFCARG | |
| | GLTHYSGSTYEEGFDFWGQGTTLTVSS | |
| HC Constant | AKTTPPSVYPLAPGSAAQTNSMVTLGCLVKGYFPEPVTVTWNSGSLSSG | 11 |
| | VHTFPAVLQSDLYTLSSSVTVPSSTWPSETVTCNVAHPASSTKVDKKIVP | |
| | RDCGCKPCICTVPEVSSVFIFPPKPKDVLTITLTPKVTCVVVDISKDDPEV | |
| | QFSWFVDDVEVHTAQTQPREEQFNSTFRSVSELPIMHQDWLNGKEFKCR | |
| | VNSAAFPAPIEKTISKTKGRPKAPQVYTIPPPKEQMAKDKVSLTCMITDFF | |
| | PEDITVEWQWNGQPAENYKNTQPIMDTDGSYFVYSKLNVQKSNWEAGN | |
| | TFTCSVLHEGLHNHHTEKSLSHSPGK | |
| Light Chain | QAVVTQESALTTSPGETVTLTCRSSTGAVTTTNYANWVQEKPDHLFTGLI | 12 |
| | GGTNNRAPGVPARFSGSLIGDKAALTITGAQTDDEAIYFCALWYSNRWV | |
| | FGGGTKLTVLGQPKSSPSVTLFPPSSEELETNKATLVCTITDFYPGVVTVD | |
| | WKVDGTPVTQGMETTQPSKQSNNKYMASSYLTLTARAWERHSSYSCQV | |
| | THEGHTVEKSLSRADCS | |
| Light Chain | MAWISLILSLLALSSGAISQAVVTQESALTTSPGETVTLTCRSSTGAVTTT | 13 |
| with leader | NYANWVQEKPDHLFTGLIGGTNNRAPGVPARFSGSLIGDKAALTITGAQ | |
| sequence | TDDEAIYFCALWYSNRWVFGGGTKLTVLGQPKSSPSVTLFPPSSEELETN | |
| | KATLVCTITDFYPGVVTVDWKVDGTPVTQGMETTQPSKQSNNKYMASS | |
| | YLTLTARAWERHSSYSCQVTHEGHTVEKSLSRADCS | |
| VL CDR1 | RSSTGAVTTTNYAN | 14 |
| VL CDR2 | GTNNRAP | 15 |
| VL CDR3 | ALWYSNRWV | 16 |
| VL FR1 | QAVVTQESALTTSPGETVTLTC | 17 |
| VL FR2 | WVQEKPDHLFTGLIG | 18 |
| VL FR3 | GVPARFSGSLIGDKAALTITGAQTDDEAIYFC | 19 |
| VL FR4 | FGGGTKLTVL | 20 |
| LC Variable | QAVVTQESALTTSPGETVTLTCRSSTGAVTTTNYANWVQEKPDHLFTGLI | 21 |
| | GGTNNRAPGVPARFSGSLIGDKAALTITGAQTDDEAIYFCALWYSNRWV | |
| | FGGGTKLTVL | |
| LC Constant | GQPKSSPSVTLFPPSSEELETNKATLVCTITDFYPGVVTVDWKVDGTPVT | 22 |
| | QGMETTQPSKQSNNKYMASSYLTLTARAWERHSSYSCQVTHEGHTVEK | |
| | SLSRADCS | |

*This sequence may include a stop sequence, such as TGA
Leader sequence is underlined.

TABLE 2
Anti-NGly-1 Antibody Nucleic Acid Sequences

| Antibody | | SEQ ID |
|---|---|---|
| Component | Nucleic Acid Sequence | NO: |
| Heavy Chain | CAGGTCCAACTGCAGCAGTCTGGAGCTGAACTGGTAAGGCCGGGGAC | 23 |
| | TTCAGTGAAGGTGTCCTGCAAGGCTTCTGGATACGCCTTCATTAATTA | |
| | CTTGATAGAATGGGTGAAACAGAGGCCTGGACAGGGCCTTGAGTGGA | |
| | TTGGAATAATAAGTCCTGGAAGTGGTAGTACTAACTACAATGAAGAG | |
| | TTCAGGGGCAGGGCAACACTGACTGCAGACAGATCCTCCAGCACTGC | |
| | CTACATGCAGCTCAGCAGCCTGACATCTGATGACTCTGCGGTCTATTT | |
| | CTGTGCAAGAGGGGGCTTAACTCATTACTCCGGCAGTACGTACGAAG | |
| | AGGGCTTTGACTTCTGGGGCCAAGGCACCACTCTCACAGTCTCCTCAG | |
| | CCAAAACGACACCCCCATCTGTCTATCCACTGGCCCCTGGATCTGCTG | |
| | CCCAAACTAACTCCATGGTGACCCTGGGATGCCTGGTCAAGGGCTATT | |
| | TCCCTGAGCCAGTGACAGTGACCTGGAACTCTGGATCCCTGTCCAGCG | |
| | GTGTGCACACCTTCCCAGCTGTCCTGCAGTCTGACCTCTACACTCTGA | |
| | GCAGCTCAGTGACTGTCCCCTCCAGCACCTGGCCCAGCGAGACCGTC | |
| | ACCTGCAACGTTGCCCACCCGGCCAGCAGCACCAAGGTGGACAAGAA | |
| | AATTGTGCCCAGGGATTGTGGTTGTAAGCCTTGCATATGTACAGTCCC | |
| | AGAAGTATCATCTGTCTTCATCTTCCCCCCAAAGCCCAAGGATGTGCT | |
| | CACCATTACTCTGACTCCTAAGGTCACGTGTGTTGTGGTAGACATCAG | |
| | CAAGGATGATCCCGAGGTCCAGTTCAGCTGGTTTGTAGATGATGTGG | |
| | AGGTGCACACAGCTCAGACGCAACCCCGGGAGGAGCAGTTCAACAGC | |
| | ACTTTCCGCTCAGTCAGTGAACTTCCCATCATGCACCAGGACTGGCTC | |
| | AATGGCAAGGAGTTCAAATGCAGGGTCAACAGTGCAGCTTTCCCTGC | |
| | CCCCATCGAGAAAACCATCTCCAAAACCAAAGGCAGACCGAAGGCTC | |
| | CACAGGTGTACACCATTCCACCTCCCAAGGAGCAGATGGCCAAGGAT | |
| | AAAGTCAGTCTGACCTGCATGATAACAGACTTCTTCCCTGAAGACATT | |
| | ACTGTGGAGTGGCAGTGGAATGGGCAGCCAGCGGAGAACTACAAGA | |
| | ACACTCAGCCCATCATGGACACAGATGGCTCTTACTTCGTCTACAGCA | |
| | AGCTCAATGTGCAGAAGAGCAACTGGGAGGCAGGAAATACTTTCACC | |
| | TGCTCTGTGTTACATGAGGGCCTGCACAACCACCATACTGAGAAGAG | |
| | CCTCTCCCACTCTCCTGGTAAA* | |
| Heavy Chain | ATGGAATGGAGCGGAGTCTTTATCTTTCTCCTGTCAGTAACTGCAGGT | 24 |
| with leader | ATTCACTCCCAGGTCCAACTGCAGCAGTCTGGAGCTGAACTGGTAAG | |
| sequence | GCCGGGGACTTCAGTGAAGGTGTCCTGCAAGGCTTCTGGATACGCCTT | |
| | CATTAATTACTTGATAGAATGGGTGAAACAGAGGCCTGGACAGGGCC | |
| | TTGAGTGGATTGGAATAATAAGTCCTGGAAGTGGTAGTACTAACTAC | |
| | AATGAAGAGTTCAGGGGCAGGGCAACACTGACTGCAGACAGATCCTC | |
| | CAGCACTGCCTACATGCAGCTCAGCAGCCTGACATCTGATGACTCTGC | |
| | GGTCTATTTCTGTGCAAGAGGGGGCTTAACTCATTACTCCGGCAGTAC | |
| | GTACGAAGAGGGCTTTGACTTCTGGGGCCAAGGCACCACTCTCACAG | |
| | TCTCCTCAGCCAAAACGACACCCCCATCTGTCTATCCACTGGCCCCTG | |
| | GATCTGCTGCCCAAACTAACTCCATGGTGACCCTGGGATGCCTGGTCA | |
| | AGGGCTATTTCCCTGAGCCAGTGACAGTGACCTGGAACTCTGGATCCC | |
| | TGTCCAGCGGTGTGCACACCTTCCCAGCTGTCCTGCAGTCTGACCTCT | |
| | ACACTCTGAGCAGCTCAGTGACTGTCCCCTCCAGCACCTGGCCCAGCG | |
| | AGACCGTCACCTGCAACGTTGCCCACCCGGCCAGCAGCACCAAGGTG | |
| | GACAAGAAAATTGTGCCCAGGGATTGTGGTTGTAAGCCTTGCATATG | |
| | TACAGTCCCAGAAGTATCATCTGTCTTCATCTTCCCCCCAAAGCCCAA | |
| | GGATGTGCTCACCATTACTCTGACTCCTAAGGTCACGTGTGTTGTGGT | |
| | AGACATCAGCAAGGATGATCCCGAGGTCCAGTTCAGCTGGTTTGTAG | |
| | ATGATGTGGAGGTGCACACAGCTCAGACGCAACCCCGGGAGGAGCA | |
| | GTTCAACAGCACTTTCCGCTCAGTCAGTGAACTTCCCATCATGCACCA | |
| | GGACTGGCTCAATGGCAAGGAGTTCAAATGCAGGGTCAACAGTGCAG | |
| | CTTTCCCTGCCCCCATCGAGAAAACCATCTCCAAAACCAAAGGCAGA | |
| | CCGAAGGCTCCACAGGTGTACACCATTCCACCTCCCAAGGAGCAGAT | |
| | GGCCAAGGATAAAGTCAGTCTGACCTGCATGATAACAGACTTCTTCC | |
| | CTGAAGACATTACTGTGGAGTGGCAGTGGAATGGGCAGCCAGCGGAG | |
| | AACTACAAGAACACTCAGCCCATCATGGACACAGATGGCTCTTACTT | |
| | CGTCTACAGCAAGCTCAATGTGCAGAAGAGCAACTGGGAGGCAGGA | |
| | AATACTTTCACCTGCTCTGTGTTACATGAGGGCCTGCACAACCACCAT | |
| | ACTGAGAAGAGCCTCTCCCACTCTCCTGGTAAA* | |
| VH CDR1 | AATTACTTGATAGAA | 25 |
| VH CDR2 | ATAATAAGTCCTGGAAGTGGTAGTACTAACTACAATGAAGAGTTCAG | 26 |
| | GGGC | |
| VH CDR3 | GGGGGCTTAACTCATTACTCCGGCAGTACGTACGAAGAGGGCTTTGA | 27 |
| | CTTC | |
| VH FR1 | CAGGTCCAACTGCAGCAGTCTGGAGCTGAACTGGTAAGGCCGGGGAC | 28 |
| | TTCAGTGAAGGTGTCCTGCAAGGCTTCTGGATACGCCTTCATT | |
| VH FR2 | TGGGTGAAACAGAGGCCTGGACAGGGCCTTGAGTGGATTGGA | 29 |
| VH FR3 | AGGGCAACACTGACTGCAGACAGATCCTCCAGCACTGCCTACATGCA | 30 |
| | GCTCAGCAGCCTGACATCTGATGACTCTGCGGTCTATTTCTGTGCAAG | |
| | A | |
| VH FR4 | TGGGGCCAAGGCACCACTCTCACAGTCTCCTCA | 31 |
| HC Variable | CAGGTCCAACTGCAGCAGTCTGGAGCTGAACTGGTAAGGCCGGGGAC | 32 |
| | TTCAGTGAAGGTGTCCTGCAAGGCTTCTGGATACGCCTTCATTAATTA | |
| | CTTGATAGAATGGGTGAAACAGAGGCCTGGACAGGGCCTTGAGTGGA | |
| | TTGGAATAATAAGTCCTGGAAGTGGTAGTACTAACTACAATGAAGAG | |
| | TTCAGGGGCAGGGCAACACTGACTGCAGACAGATCCTCCAGCACTGC | |
| | CTACATGCAGCTCAGCAGCCTGACATCTGATGACTCTGCGGTCTATTT | |
| | CTGTGCAAGAGGGGGCTTAACTCATTACTCCGGCAGTACGTACGAAG | |
| | AGGGCTTTGACTTCTGGGGCCAAGGCACCACTCTCACAGTCTCCTCA | |
| HC Constant | GCCAAAACGACACCCCCATCTGTCTATCCACTGGCCCCTGGATCTGCT | 33 |
| | GCCCAAACTAACTCCATGGTGACCCTGGGATGCCTGGTCAAGGGCTA | |
| | TTTCCCTGAGCCAGTGACAGTGACCTGGAACTCTGGATCCCTGTCCAG | |
| | CGGTGTGCACACCTTCCCAGCTGTCCTGCAGTCTGACCTCTACACTCT | |
| | GAGCAGCTCAGTGACTGTCCCCTCCAGCACCTGGCCCAGCGAGACCG | |
| | TCACCTGCAACGTTGCCCACCCGGCCAGCAGCACCAAGGTGGACAAG | |
| | AAAATTGTGCCCAGGGATTGTGGTTGTAAGCCTTGCATATGTACAGTC | |
| | CCAGAAGTATCATCTGTCTTCATCTTCCCCCCAAAGCCCAAGGATGTG | |
| | CTCACCATTACTCTGACTCCTAAGGTCACGTGTGTTGTGGTAGACATC | |
| | AGCAAGGATGATCCCGAGGTCCAGTTCAGCTGGTTTGTAGATGATGT | |
| | GGAGGTGCACACAGCTCAGACGCAACCCCGGGAGGAGCAGTTCAAC | |
| | AGCACTTTCCGCTCAGTCAGTGAACTTCCCATCATGCACCAGGACTGG | |
| | CTCAATGGCAAGGAGTTCAAATGCAGGGTCAACAGTGCAGCTTTCCC | |
| | TGCCCCCATCGAGAAAACCATCTCCAAAACCAAAGGCAGACCGAAGG | |
| | CTCCACAGGTGTACACCATTCCACCTCCCAAGGAGCAGATGGCCAAG | |
| | GATAAAGTCAGTCTGACCTGCATGATAACAGACTTCTTCCCTGAAGAC | |
| | ATTACTGTGGAGTGGCAGTGGAATGGGCAGCCAGCGGAGAACTACAA | |
| | GAACACTCAGCCCATCATGGACACAGATGGCTCTTACTTCGTCTACAG | |
| | CAAGCTCAATGTGCAGAAGAGCAACTGGGAGGCAGGAAATACTTTCA | |
| | CCTGCTCTGTGTTACATGAGGGCCTGCACAACCACCATACTGAGAAG | |
| | AGCCTCTCCCACTCTCCTGGTAAA* | |
| Light Chain | CAGGCTGTTGTGACTCAGGAATCTGCACTCACCACATCACCTGGTGAA | 34 |
| | ACAGTCACACTCACTTGTCGCTCAAGTACTGGGGCTGTTACAACTACT | |
| | AACTATGCCAACTGGGTCCAAGAAAAACCAGATCATTTATTCACTGG | |
| | TCTAATAGGTGGTACCAACAACCGAGCTCCAGGTGTTCCTGCCAGATT | |
| | CTCAGGCTCCCTGATTGGAGACAAGGCTGCCCTCACCATCACAGGGG | |
| | CACAGACTGATGATGAGGCAATATATTTCTGTGCTCTATGGTACAGCA | |
| | ACCGCTGGGTGTTCGGTGGAGGAACCAAACTGACTGTCCTAGGCCAG | |
| | CCCAAGTCTTCGCCATCAGTCACCCTGTTTCCACCTTCCTCTGAAGAG | |
| | CTCGAGACTAACAAGGCCACACTGGTGTGTACGATCACTGATTTCTAC | |
| | CCAGGTGTGGTGACAGTGGACTGGAAGGTAGATGGTACCCCTGTCAC | |
| | TCAGGGTATGGAGACAACCCAGCCTTCCAAACAGAGCAACAACAAGT | |
| | ACATGGCTAGCAGCTACCTGACCCTGACAGCAAGAGCATGGGAAAGG | |
| | CATAGCAGTTACAGCTGCCAGGTCACTCATGAAGGTCACACTGTGGA | |
| | GAAGAGTTTGTCCCGTGCTGACTGTTCC* | |
| Light Chain | ATGGCCTGGATTTCACTTATACTCTCTCTCCTGGCTCTCAGCTCAGGG | 35 |
| with leader | GCCATTTCCCAGGCTGTTGTGACTCAGGAATCTGCACTCACCACATCA | |
| sequence | CCTGGTGAAACAGTCACACTCACTTGTCGCTCAAGTACTGGGGCTGTT | |
| | ACAACTACTAACTATGCCAACTGGGTCCAAGAAAAACCAGATCATTT | |
| | ATTCACTGGTCTAATAGGTGGTACCAACAACCGAGCTCCAGGTGTTCC | |
| | TGCCAGATTCTCAGGCTCCCTGATTGGAGACAAGGCTGCCCTCACCAT | |
| | CACAGGGGCACAGACTGATGATGAGGCAATATATTTCTGTGCTCTAT | |
| | GGTACAGCAACCGCTGGGTGTTCGGTGGAGGAACCAAACTGACTGTC | |
| | CTAGGCCAGCCCAAGTCTTCGCCATCAGTCACCCTGTTTCCACCTTCC | |
| | TCTGAAGAGCTCGAGACTAACAAGGCCACACTGGTGTGTACGATCAC | |
| | TGATTTCTACCCAGGTGTGGTGACAGTGGACTGGAAGGTAGATGGTA | |
| | CCCCTGTCACTCAGGGTATGGAGACAACCCAGCCTTCCAAACAGAGC | |
| | AACAACAAGTACATGGCTAGCAGCTACCTGACCCTGACAGCAAGAGC | |
| | ATGGGAAAGGCATAGCAGTTACAGCTGCCAGGTCACTCATGAAGGTC | |
| | ACACTGTGGAGAAGAGTTTGTCCCGTGCTGACTGTTCC* | |
| VL CDR1 | CGCTCAAGTACTGGGGCTGTTACAACTACTAACTATGCCAAC | 36 |
| VL CDR2 | GGTACCAACAACCGAGCTCCA | 37 |
| VL CDR3 | GCTCTATGGTACAGCAACCGCTGGGTG | 38 |
| VL FR1 | CAGGCTGTTGTGACTCAGGAATCTGCACTCACCACATCACCTGGTGAA | 39 |
| | ACAGTCACACTCACTTGT | |
| VL FR2 | TGGGTCCAAGAAAAACCAGATCATTTATTCACTGGTCTAATAGGT | 40 |
| VL FR3 | GGTGTTCCTGCCAGATTCTCAGGCTCCCTGATTGGAGACAAGGCTGCC | 41 |
| | CTCACCATCACAGGGGCACAGACTGATGATGAGGCAATATATTTCTG | |
| | T | |
| VL FR4 | TTCGGTGGAGGAACCAAACTGACTGTCCTA | 42 |
| LC Variable | CAGGCTGTTGTGACTCAGGAATCTGCACTCACCACATCACCTGGTGAA | 43 |
| | ACAGTCACACTCACTTGTCGCTCAAGTACTGGGGCTGTTACAACTACT | |
| | AACTATGCCAACTGGGTCCAAGAAAAACCAGATCATTTATTCACTGG | |
| | TCTAATAGGTGGTACCAACAACCGAGCTCCAGGTGTTCCTGCCAGATT | |
| | CTCAGGCTCCCTGATTGGAGACAAGGCTGCCCTCACCATCACAGGGG | |
| | CACAGACTGATGATGAGGCAATATATTTCTGTGCTCTATGGTACAGCA | |
| | ACCGCTGGGTGTTCGGTGGAGGAACCAAACTGACTGTCCTA | |
| LC Constant | GGCCAGCCCAAGTCTTCGCCATCAGTCACCCTGTTTCCACCTTCCTCT | 44 |
| | GAAGAGCTCGAGACTAACAAGGCCACACTGGTGTGTACGATCACTGA | |
| | TTTCTACCCAGGTGTGGTGACAGTGGACTGGAAGGTAGATGGTACCC | |
| | CTGTCACTCAGGGTATGGAGACAACCCAGCCTTCCAAACAGAGCAAC | |
| | AACAAGTACATGGCTAGCAGCTACCTGACCCTGACAGCAAGAGCATG | |
| | GGAAAGGCATAGCAGTTACAGCTGCCAGGTCACTCATGAAGGTCACA | |
| | CTGTGGAGAAGAGTTTGTCCCGTGCTGACTGTTCC* | |

*This sequence may include a stop sequence, such as TGA
Leader sequence is underlined.

### Example 2

This example provides a comparison of the anti-NGly-1 antibody of the present disclosure to the previously-described and/or commercially-available anti-NGly-1 antibodies listed in Table 3.

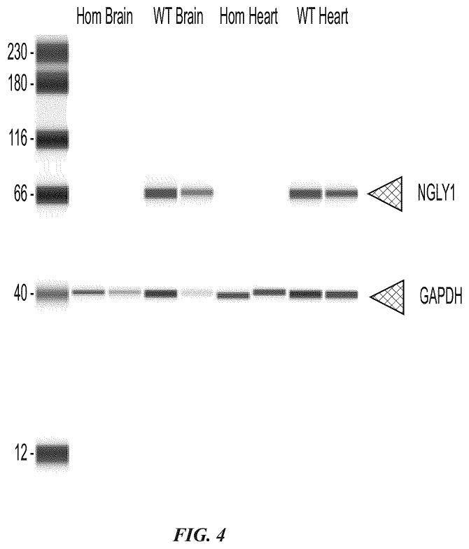

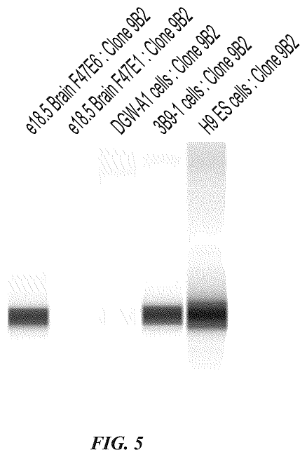

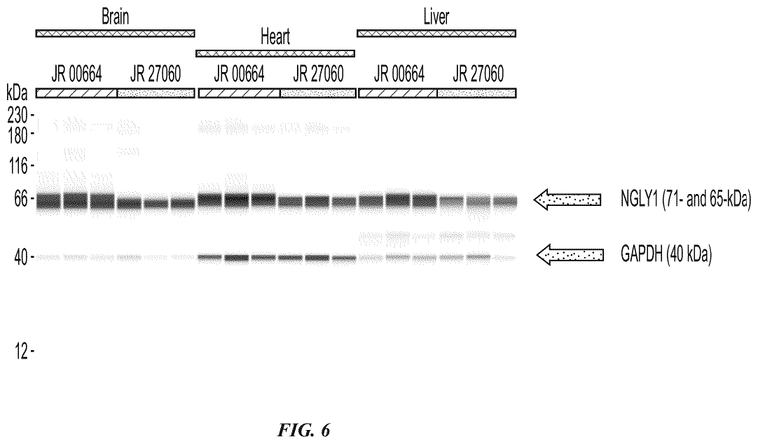

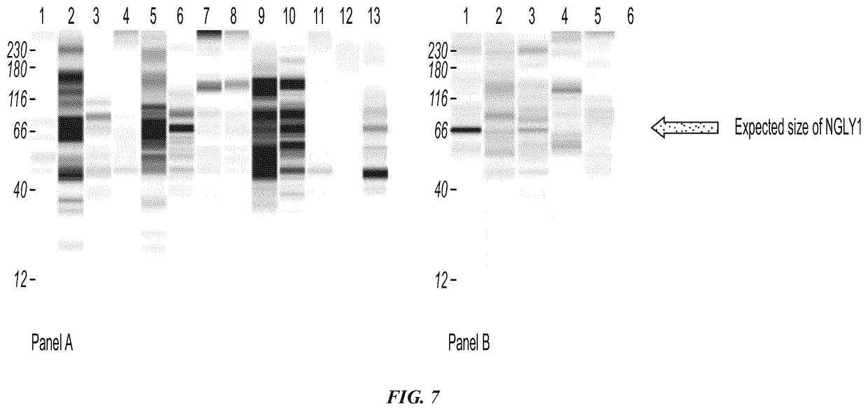

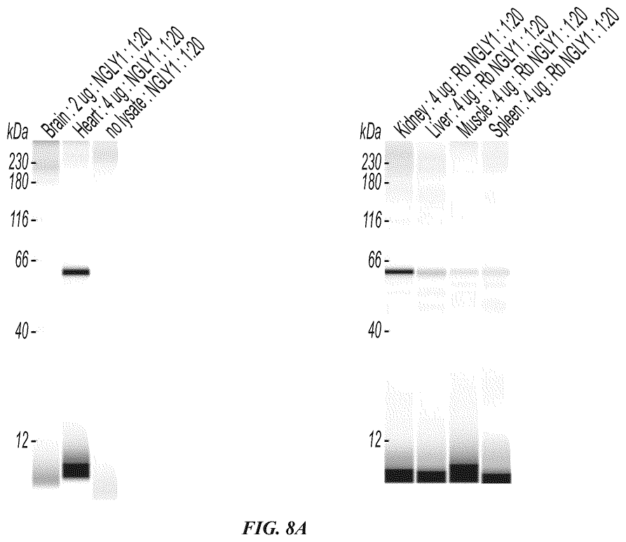

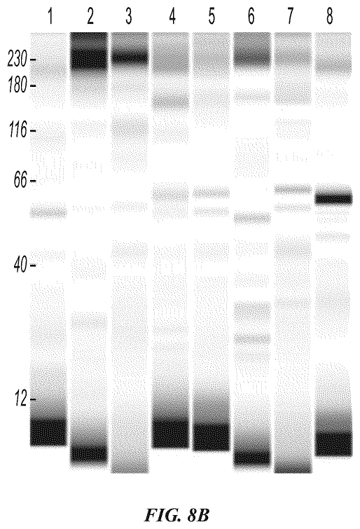

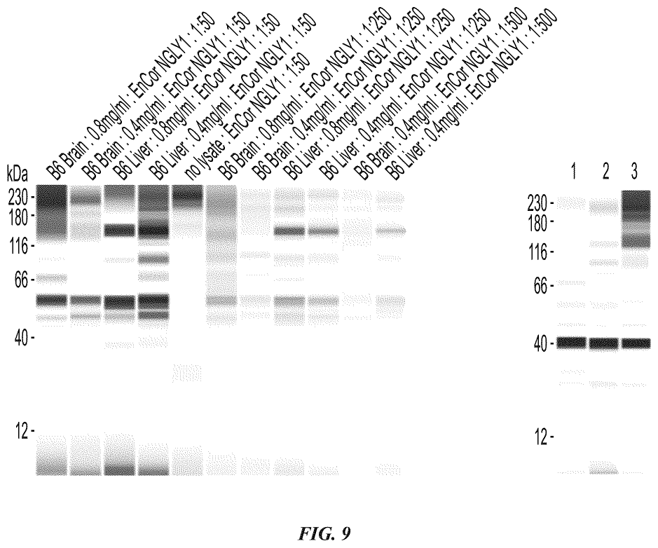

FIGS. 4-9 describe the conditions used and results obtained.

TABLE 3
Antibodies Tested: NGly-1 Specificity

| | MW of | | | |
|---|---|---|---|---|
| Source | protein | Dilution | Source | Cat # |
| Mouse | 66 kDa | 1:2000 | JAX | |
| monoclonal | | (tissues) | Monoclonal | |
| (anti-mouse | | 1:2000 | Service | |
| peptide) | | (cell lines) | | |
| Rabbit | 66 kDa | 1:400 | Gift from Dr. | Riken.jp |
| polyclonal | | | Tadashi Suzuki | |
| Rabbit | 74 kDa | 1:50 | SIGMA- | HPA036825 |
| polyclonal | | (tissues) | ALDRICH | |
| | | 1:100 | | |
| | | (cell lines) | | |
| Rabbit | 74 kDa | 1:20 | ABCAM | ab197107 |
| polyclonal | | (tissues) | | |
| (anti-human | | 1:100 | | |
| amino acids | | (cell line) | | |
| 327-538) | | | | |
| Rabbit | 66 kDa | 1:250 | EnCor | RPCA- |
| polyclonal | | (tissues) | Biotechnology | NGLY1 |
| (anti human | | 1:250 | | |
| amino acids | | (cell lines) | | |
| 1-300) | | | | |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean +10% of the recited numerical value.

Where a range of values is provided, each value between the upper and lower ends of the range are specifically contemplated and described herein.

1. An antibody that binds specifically to N-glycanase 1 and comprises a heavy chain and a light chain, wherein
the heavy chain comprises
(a) a CDR1 comprising the sequence of SEQ ID NO: 3,
(b) a CDR2 comprising the sequence of SEQ ID NO: 4, and
(c) a CDR3 comprising the sequence of SEQ ID NO: 5, and/or
the light chain comprises
(a) a CDR1 comprising the sequence of SEQ ID NO: 14,
(b) a CDR2 comprising the sequence of SEQ ID NO: 15, and
(c) a CDR3 comprising the sequence of SEQ ID NO: 16.

2.-38. (canceled)

39. A polynucleotide encoding the antibody of claim 1.

40.-43. (canceled)

44. A method comprising administering to a subject an antibody that binds specifically to N-glycanase 1, wherein the antibody comprises:
a heavy chain comprising
(a) a CDR1 comprising the sequence of SEQ ID NO: 3,
(b) a CDR2 comprising the sequence of SEQ ID NO: 4, and
(c) a CDR3 comprising the sequence of SEQ ID NO: 5, and
a light chain comprising
(a) a CDR1 comprising the sequence of SEQ ID NO: 14,
(b) a CDR2 comprising the sequence of SEQ ID NO: 15, and
(c) a CDR3 comprising the sequence of SEQ ID NO: 16.

45. The method of claim 44, wherein the subject has a cancer.

46. The method of claim 45, wherein the cancer is selected from the group consisting of melanoma, ovarian cancer, breast cancer, cervical cancer, endometrial cancer, colon cancer, pancreatic cancer, prostate cancer, liver cancer, renal cancer, lung cancer, glioma, and lymphoma.

47. (canceled)

48. A method of diagnosing or monitoring the progression of a N-glycanase 1 (NGLY-1) deficiency in a subject, the method comprising
assessing a level of NGLY-1 protein in a biological sample from a subject with an anti-NGLY-1 antibody of claim 1, wherein the equilibrium dissociation constant (K_{D}) between the anti-NGLY-1 antibody and NGLY-1 protein is 100 pM to 500 pM; and
comparing the level of NGLY-1 protein to a reference standard indicative of a level NGLY-1 protein in a control subject who does not have a NGLY-1 deficiency or a control subject who does have a NGLY-1 deficiency, wherein a difference or similarity between the level of NGLY-1 protein and the reference standard is indicative that the subject has a NGLY-1 deficiency.

49. A method of monitoring the efficacy of a N-glycanase 1 (NGLY-1) deficiency therapy in a subject, the method comprising
assessing a level of NGLY-1 protein in a biological sample from a subject undergoing therapy for a NGLY-1 deficiency with an anti-NGLY-1 antibody of claim 1, wherein the equilibrium dissociation constant (K_{D}) between the anti-NGLY-1 antibody and NGLY-1 protein is 100 pM to 500 pM; and
comparing the level of NGLY-1 protein to a reference standard indicative of a level NGLY-1 protein in a control subject who does not have a NGLY-1 deficiency or a control subject who does have a NGLY-1 deficiency, wherein a difference or similarity between the level of NGLY-1 protein and the reference standard is indicative of efficacy of the therapy.

50.-57. (canceled)

58. The method of claim 44, wherein the heavy chain comprises: a framework region that is at least 90% or at least 95% identical to the sequence of SEQ ID NO: 6; a framework region that is at least 90% or at least 95% identical to the sequence of SEQ ID NO: 7; a framework region that is at least 90% or at least 95% identical to the sequence of SEQ ID NO: 8; and/or a framework region that is at least 90% or at least 95% identical to the sequence of SEQ ID NO:9.

59. The method of claim 44, wherein the heavy chain comprises a variable region that is at least 90% or at least 95% identical to the sequence of SEQ ID NO: 10.

60. The method of claim 44, wherein the heavy chain comprises a constant region that is at least 90% or at least 95% identical to the sequence of SEQ ID NO: 11.

61. The method of claim 44, wherein the heavy chain comprises the sequence of SEQ ID NO: 1.

62. The method of claim 44, wherein the light chain comprises: a framework region that is at least 90% identical to the sequence of SEQ ID NO: 17; a framework region that is at least 90% identical to the sequence of SEQ ID NO: 18; a framework region that is at least 90% identical to the sequence of SEQ ID NO: 19; and/or a framework region that is at least 90% identical to the sequence of SEQ ID NO: 20.

63. The method of claim 44, wherein the light chain comprises a variable region that is at least 90% or at least 95% identical to the sequence of SEQ ID NO: 21.

64. The method of claim 44, wherein the light chain comprises a constant region that is at least 90% or at least 95% identical to the sequence of SEQ ID NO: 22.

65. The method of claim 44, wherein the light chain comprises the sequence of SEQ ID NO: 12.
